SEQUENCE LISTING (5 sequences)

SEQ ID NO: 1 - protein, 594 aa, Arabidopsis thaliana
MQKRIALSFPEEVLEHVFSFIQLDKDRNSVSLVCKSWYEIERWCRRKVFIGNCYAVSPATVIRRFPKVRSVELKGKPHFADFNLVPDGWGGYVYPWIEAMSSSYTWLEEIRLKRMVVTDDCLELIAKSFKNFKVLVLSSCEGFSTDGLAAIAATCRNLKELDLRESDVDDVSGHWLSHFPDTYTSLVSLNISCLASEVSFSALERLVTRCPNLKSLKLNRAVPLEKLATLLQRAPQLEELGTGGYTAEVRPDVYSGLSVALSGCKELRCLSGFWDAVPAYLPAVYSVCSRLTTLNLSYATVQSYDLVKLLCQCPKLQRLWVLDYIEDAGLEVLASTCKDLRELRVFPSEPFVMEPNVALTEQGLVSVSMGCPKLESVLYFCRQMTNAALITIARNRPNMTRFRLCIIEPKAPDYLTLEPLDIGFGAIVEHCKDLRRLSLSGLLTDKVFEYIGTYAKKMEMLSVAFAGDSDLGMHHVLSGCDSLRKLEIRDCPFGDKALLANASKLETMRSLWMSSCSVSFGACKLLGQKMPKLNVEVIDERGAPDSRPESCPVERVFIYRTVAGPRFDMPGFVWNMDQDSTMRFSRQIITTNGL

SEQ ID NO: 2 - DNA, 1785 bp, Arabidopsis thaliana
  source 1..1785: mol_type = other DNA
atgcagaagcgaatagccttgtcgtttccagaagaggtactagagcatgtgttctcgtttattcagctggataaggataggaactcagtctctctggtgtgcaagtcatggtacgagatcgagcggtggtgcaggaggaaagtcttcatcgggaactgctacgccgtgagtccagcgacggtgattaggaggttcccgaaagtgagatccgtggagcttaaaggaaaacctcactttgctgactttaatttggtacctgacggatggggaggttacgtgtatccatggattgaggccatgtcttcgtcttacacgtggcttgaagagataaggctgaagaggatggtggtcaccgacgattgcttggagctcatagccaagtcttttaagaattttaaggttcttgtgctttcttcctgcgaaggcttctccaccgatggtctcgctgctatcgctgccacttgcaggaatctgaaagagcttgacttacgagagagtgatgttgacgacgttagtggccactggcttagccatttcccagatacatacacttctttggtatcactcaatatatcttgcttagcatctgaggtcagtttctctgctctggaaaggctggtgactaggtgtcccaatctcaagtctctcaagcttaaccgagctgttccacttgaaaaattggctactttacttcaaagagcacctcaattggaggaattgggcactggtgggtacactgcagaagtgcgaccagatgtttactctggtttatctgtagcgctctctgggtgcaaggaattgaggtgcttatctggattttgggatgctgttcctgcctatcttccagcagtttattcggtttgcagtcggcttacaactttgaatctgagttatgcaacagtccagagctatgatcttgtcaagcttctttgtcaatgccctaaactgcagcgcctctgggtgcttgactacatcgaggatgctggtcttgaggtgcttgcttcaacctgcaaggacctacgcgagctgagagtgtttccgtccgagccttttgtcatggaaccaaatgtggcattgacggaacaggggcttgtctccgtctccatgggctgtccaaaactcgagtcggttctctacttctgccgtcaaatgaccaatgctgcattgataaccattgctaggaaccgtcccaacatgactcgcttccgtttgtgcatcattgagccaaaagccccagactatctgactctagagccactggatattggatttggagccatagtagagcactgcaaggatctccgtcgcctctctctatctggcctcttgaccgacaaggtttttgaatacattgggacatatgccaagaagatggaaatgctctcagtggcatttgcaggagacagtgacttaggcatgcatcatgttttgtccgggtgcgatagcttgaggaaactagagataagggactgcccgtttggagacaaggcgcttttggccaatgcttcaaagctggagacaatgcgatccctttggatgtcttcttgttccgtgagttttggagcctgcaagttactaggacagaagatgccaaagctgaatgtggaagtcatcgatgaacggggtgcaccggactcgagaccagagagctgccctgttgagagagtcttcatataccgaacagtggctggtcctcgatttgacatgcctggcttcgtctggaacatggaccaagactcaacaatgaggttttccaggcaaatcattactactaacggattataa

SEQ ID NO: 3 - protein, 594 aa, Arabidopsis thaliana
MQKRIALSFPEEVLEHVFSFIQLDKDRNSVSLVCKSWYEIERWCRRKVFIGNCYAVSPATVIRRFPKVRSVELKGKPHGADFNLVPDGWGGYVYPWIEAMSSSYTWLEEIRLKRMVVTDDCLELIAKSFKNFKVLVLSSCEGFSTDGLAAIAATCRNLKELDLRESDVDDVSGHWLSHFPDTYTSLVSLNISCLASEVSFSALERLVTRCPNLKSLKLNRAVPLEKLATLLQRAPQLEELGTGGYTAEVRPDVYSGLSVALSGCKELRCLSGFWDAVPAYLPAVYSVCSRLTTLNLSYATVQSYDLVKLLCQCPKLQRLWVLDYIEDAGLEVLASTCKDLRELRVFPSEPFVMEPNVALTEQGLVSVSMGCPKLESVLYFCRQMTNAALITIARNRPNMTRFRLCIIEPKAPDYLTLEPLDIGFGAIVEHCKDLRRLSLSGLLTDKVFEYIGTYAKKMEMLSVAFAGDSDLGMHHVLSGCDSLRKLEIRDCPFGDKALLANASKLETMRSLWMSSCSVSFGACKLLGQKMPKLNVEVIDERGAPDSRPESCPVERVFIYRTVAGPRFDMPGFVWNMDQDSTMRFSRQIITTNGL

SEQ ID NO: 4 - protein, 68 aa, synthetic construct
  REGION 1..68: note = Mini auxin inducible degron
KEKSACPKDPAKPPAKAQVVGWPPVRSYRKNVMVSCQKSSGGPEAAAFVKVSMDGAPYLRKIDLRMYK

SEQ ID NO: 5 - protein, 44 aa, synthetic construct
  REGION 1..44: note = AID*
PKDPAKPPAKAQVVGWPPVRSYRKNVMVSCQKSSGGPEAAAFVK

(19) United States
(12) Patent Application Publication
(10) Pub. No.: US 2025/0122250 A1
(43) Pub. Date: Apr. 17, 2025

(54) AUXIN-DEGRON SYSTEM KIT AND USE THEREFOR

(71) Applicants: Inter-University Research Institute Corporation Research Organization of Information and Systems, Tokyo (JP); Kake Educational Institution, Okayama-shi, Okayama (JP)

(72) Inventors: Masato Kanemaki, Mishima-shi (JP); Ken-ichiro Hayashi, Okayama-shi (JP); Takefumi Negishi, Mishima-shi (JP)

(73) Assignees: Inter-University Research Institute Corporation Research Organization of Information and Systems, Tokyo (JP); Kake Educational Institution, Okayama-shi, Okayama (JP)

(21) Appl. No.: 18/685,477

(22) PCT Filed: Aug. 5, 2022
(86) PCT No.: PCT/JP2022/030115

§ 371 Date: Feb. 21, 2024

(30) Foreign Application Priority Data
Aug. 23, 2021 (JP) 2021-135637

(51) Int. Cl. C07K 14/415; C07D 209/18

(52) U.S. Cl. CPC C07K 14/415; C07D 209/18; C07K 2319/95

(57) ABSTRACT

An auxin-degron system kit controlling degradation of a target protein in a non-plant-derived eukaryotic cell, in which the auxin-degron system kit includes a first nucleic acid encoding a mutant TIR1 family protein that has a mutation at an auxin-binding site; an auxin analog having an affinity for the mutant TIR1 family protein; and a second nucleic acid encoding a degradation tag that includes at least a partial Aux/IAA family protein and has an affinity for a complex of the mutant TIR1 family protein and the auxin analog.

### TECHNICAL FIELD

The present invention relates to an auxin-degron system kit and a use therefor. Specifically, the present invention relates to an auxin-degron system kit, a method for degrading a target protein, an inducer for degradation of a target protein, and a compound.

Priority is claimed on Japanese Patent Application No. 2021-135637, filed Aug. 23, 2021, the content of which is incorporated herein by reference.

### BACKGROUND ART

The present inventors have thus far developed a protein degradation control technique called an auxin-inducible degron (AID) system. In this system, a TIR1 that constitutes an auxin-responsive ubiquitin ligase is introduced into a cell derived from a eukaryote such as a yeast and animal cells, and the degradation of a target protein to which a degradation tag (a plant-derived Aux/IAA family protein or a partial protein thereof; also referred to as a degron) has been added is controlled by adjusting the presence or absence of an auxin, or an addition timing of the auxin.

The use of an auxin-degron method in the related art makes it possible to rapidly degrade a target protein to which a degradation tag (degron) has been added, by addition of an auxin. However, in the auxin-degron method in the related art, weak degradation of a target protein occurs even without addition of the auxin. Thus, it was difficult to control the strict expression. In addition, a relatively high concentration (100 μM or higher) of the auxin is used to induce the degradation. Thus, there has been a concern about a toxic effect of the auxin itself, particularly on multicellular animals.

In response to these concerns, the present inventors have developed an improved auxin-inducible degron (AID) system (also referred to as an AID2 system) in which a mutant TIR1 and an auxin analog having an affinity for the mutant TIR1 are combined (see Patent Document 1).

### CITATION LIST

### Patent Document

### [Patent Document 1]

PCT International Publication No. WO2021/009990

### SUMMARY OF INVENTION

### Technical Problem

In the AID2 system, the degradation efficiency of a target protein has been dramatically improved, but there has been a problem in that the membrane permeability of an auxin or an auxin analog is impaired due to the barrier function in an eggshell or the blood-brain barrier. Thus, there has been a demand for improvement in terms of the problem.

The present invention has been made in view of these circumstances, and thus provides an auxin-degron system kit, a method for degrading a target protein, an inducer for degradation of a target protein, and a compound, each of which enables efficient control of degradation of a protein.

### Solution to Problem

The present invention includes the following aspects.

[1] An auxin-degron system kit controlling degradation of a target protein in a non-plant-derived eukaryotic cell, the kit including:
a first nucleic acid encoding a mutant TIR1 family protein that has a mutation at an auxin-binding site;
an auxin analog having an affinity for the mutant TIR1 family protein; and
a second nucleic acid encoding a degradation tag that includes at least a partial Aux/IAA family protein and has an affinity for a complex of the mutant TIR1 family protein and the auxin analog,
in which the mutant TIR1 family protein is a protein that consists of a sequence including any one amino acid sequence of the following (a) to (c), and binds to the degradation tag through the complex with the auxin analog, leading to degradation of the target protein,
(a) an amino acid sequence in which an amino acid at position 79 of an amino acid sequence set forth in SEQ ID NO: 1 is glycine,
(b) an amino acid sequence in which one to several amino acids are deleted, inserted, substituted, or added in a site other than the amino acid at position 79 of (a), and
(c) an amino acid sequence having 80% or more identity in a site other than the amino acid at position 79 of (a), and
the auxin analog is a compound represented by General Formula (I).

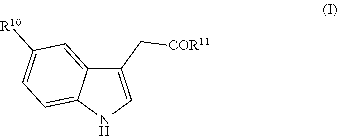

(In General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

[2] The kit according to [1], further including:
a third nucleic acid encoding a target protein, which is linked upstream or downstream of the second nucleic acid.

[3] The kit according to [1] or [2],
in which the mutant TIR1 family protein is an Arabidopsis thaliana-derived protein.

[4] The kit according to any one of [1] to [3],
in which the mutant TIR1 family protein is a protein in which F at position 79 of AtTIR1 has been mutated into A, G, or S.

[5] The kit according to any one of [1] to [4], further including:
a non-plant-derived eukaryotic cell or non-human animal, having the first nucleic acid on a chromosome.

[6] The kit according to [5],
in which the non-plant-derived eukaryotic cell or non-human animal further has a chromosome including a second nucleic acid encoding a degradation tag that includes at least a partial Aux/IAA family protein and has an affinity for a complex of the mutant TIR1 family protein and the auxin analog, and a third nucleic acid encoding a target protein, which is linked upstream or downstream of the second nucleic acid.

[7] A method for degrading a target protein, the method using the kit according to any one of [1] to [6].

[8] An inducer for degradation of a target protein, used in an auxin-degron system controlling degradation of a target protein in a non-plant-derived eukaryotic cell,
the inducer comprising:
a compound represented by General Formula (I).

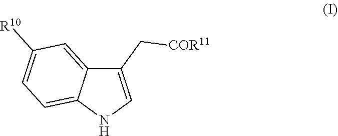

(In General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

[9] A compound represented by General Formula (I).

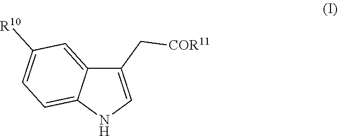

(In General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

### Advantageous Effects of Invention

According to the auxin-degron system kit of the present invention, it is possible to efficiently control degradation of a protein.

### BRIEF DESCRIPTION OF DRAWINGS

FIG. 1A shows representative images of lineages of nematodes for evaluating an AID system and an AID2 system. The genotype of each lineage is shown on the left side. All scale bars indicate 50 μm.

FIG. 1B shows the results of quantification of average intensities of GFP signals in each lineage. The error bar indicates a standard deviation.

FIG. 2 shows the results obtained by treating L1 larvae of each genotype with a ligand (1 mM IAA or 5 μM 5-Ph-IAA) or a solvent thereof, and counting the number of progeny thereof (n=10). The error bar indicates a standard deviation.

FIG. 3A shows representative images before and after a ligand treatment. Each larva was treated with 1 mM IAA (left) or 5 μM 5-Ph-IAA (right) for 1 hour. All scale bars indicate 50 μm.

FIG. 3B is a graph showing the temporal courses of average intensities of GFP in larvae treated with ligands. The error bar indicates a standard deviation.

FIG. 3C shows the results of quantification of GFP levels in larvae of each lineage treated with different concentrations of IAA or 5-Ph-IAA for one generation. The error bar indicates a standard deviation.

FIG. 3D shows representative images of reporter protein expressions in nematodes in a young adult phase expressing AtTIR1 (F79G)::mRuby after being treated with DMSO (upper panel) or 5 μM 5-Ph-IAA (lower panel) for 4 hours. The scale bar indicates 50 μm.

FIG. 3E shows the results of average intensities of GFP in nematodes in an adult phase treated with 5-Ph-IAA for 4 hours. The error bar indicates a standard deviation.

FIG. 4A is as follows:

Upper panel: Chemical structure of 5-Ph-IAA. Lower panels: Time-lapse images of an embryo (AID*::GFP; AtTIR1 (F79G)::mRuby) treated with 50 μM 5-Ph-IAA. The scale bar indicates 20 μm.

FIG. 4B is a graph showing the GFP intensities of 10 embryos (AID*::GFP; AtTIR1 (F79G)::mRuby) treated with 50 μM 5-Ph-IAA. The images were acquired at intervals of 3 min.

FIG. 4C is a graph showing the effect of 50 μM 5-Ph-IAA causing the degradation of a reporter. The GFP intensities were measured at 0 min and 60 min (n=23).

FIG. 4D is as follows:

Upper panel: Chemical structure of 5-Ph-IAA-AM. Lower panels: Time-lapse images of an embryo (AID*::GFP; AtTIR1 (F79G)::mRuby) treated with 50 μM 5-Ph-IAA. The scale bar indicates 20 μm.

FIG. 4E is a graph showing the GFP intensities of embryos (AID*::GFP; AtTIR1 (F79G)::mRuby) treated with DMSO (n=5) or 50 μM 5-Ph-IAA-AM (n=4). The images were acquired at intervals of 3 min.

FIG. 4F is a graph showing the effect of 50 μM 5-Ph-IA-AMA causing the degradation of a reporter. The GFP intensities were measured at 0 min and 60 min (n=24).

### DESCRIPTION OF EMBODIMENTS

### <<Auxin-Degron System Kit>>

The kit of the present invention is an auxin-degron system kit controlling the degradation of a target protein in a non-plant-derived eukaryotic cell, the kit including a first nucleic acid encoding a mutant TIR1 family protein that has a mutation at an auxin-binding site; an auxin analog having an affinity for the mutant TIR1 family protein; and a second nucleic acid encoding a degradation tag that includes at least a partial Aux/IAA family protein and has an affinity for a complex of the mutant TIR1 family protein and the auxin analog, in which the mutant TIR1 family protein is a protein that consists of a sequence including any one amino acid sequence of the following (a) to (c), and binds to the degradation tag through the complex with the auxin analog, leading to degradation of the target protein,
(a) an amino acid sequence in which an amino acid at position 79 of an amino acid sequence set forth in SEQ ID NO: 1 is glycine,
(b) an amino acid sequence in which one to several amino acids are deleted, inserted, substituted, or added in a site other than the amino acid at position 79 of (a), and
(c) an amino acid sequence having 80% or more identity in a site other than the amino acid at position 79 of (a), and
the auxin analog is a compound represented by General Formula (I).

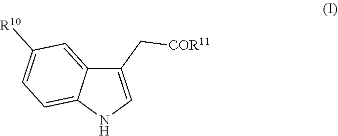

(In General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

An “auxin-degron system” is a protein degradation control technique developed by the present inventors, and is a system in which a plant-specific protein degradation system introduced by a plant hormone, an auxin, is applied to a non-plant-derived eukaryotic cell.

Specifically, this system is a system in which a plant-derived TIR1 family protein as an F-box protein, which is a subunit of an E3 ubiquitin ligase complex (SCF complex), and a target protein labeled with a peptide consisting of a plant-derived Aux/IAA family protein or a partial sequence thereof are introduced into a non-plant-derived eukaryotic cell, whereby the TIR1 family protein, which is an auxin receptor, recognizes a peptide consisting of the Aux/IAA family protein or a partial sequence thereof and degrades the target protein using a ubiquitin/proteasome degradation system in the non-plant-derived eukaryotic cell in an auxin-dependent manner.

In such a system, the present inventors have found a problem in that the membrane permeability of an auxin or an auxin analog is impaired by a barrier function in an eggshell or the blood-brain barrier. With regard to such a problem, the present inventors have found a combination of a mutant TIR1 family protein that has a mutation at an auxin-binding site, an auxin analog having an affinity for the mutant TIR1 family protein, in which the auxin analog has improved membrane permeability, and a degradation tag that includes at least a partial Aux/IAA family protein and has an affinity for a complex of the mutant TIR1 family protein and the auxin analog.

According to the present invention, it is possible to provide an auxin-degron system with an extremely high efficiency in the degradation of a target protein.

### <First Nucleic Acid>

In the present invention, the first nucleic acid encodes a mutant TIR1 family protein that has a mutation at an auxin-binding site.

The TIR1 family protein is an F-box protein which is one of subunits forming an E3 ubiquitin ligase complex (SCF complex) in protein degradation by an ubiquitin-proteasome system, and is a plant-specific protein. The TIR1 family protein serves as a receptor of an auxin, which is a growth hormone, and is known to recognize an Aux/IAA family protein, which is an inhibiting factor of an auxin communication system, by receiving an auxin, and thus to degrade a target protein.

The type of the gene encoding the TIR1 family protein is not limited as long as it is a gene encoding a plant-derived TIR1 family protein. In addition, the type of the plant from which the TIR1 family protein is derived is also not limited, and examples of the plant include Arabidopsis thaliana, rice, zinnia, pines, ferns, and Physcomitrella patens. Specific examples of the gene encoding the TIR1 family protein include a TIR1 gene, an AFB1 gene, an AFB2 gene, an AFB3 gene, an FBX14 gene, and an AFB5 gene.

Among these, an AtTIR1 gene, which is an Arabidopsis thaliana-derived TIR1 gene, or a rice-derived OsTIR1 is preferable. Examples of such a gene include a gene with an accession number NM_116163 (Gene ID: 825473) that is registered in NCBI, and more specifically, a gene consisting of a base sequence represented by SEQ ID NO: 2.

In the present invention, the mutant TIR1 family protein has a mutation at an auxin-binding site. Such a mutant protein is not particularly limited as long as it has an affinity for an auxin analog which will be described below, but is preferably a mutant protein in which the F at position 79 of AtTIR1 has been mutated into A, G, or S, and more preferably a mutant protein in which the F at position 79 has been mutated into G.

Specifically, the mutant TIR1 family protein is a protein that consists of a sequence including any one amino acid sequence of the following (a) to (c), and binds to the degradation tag through the complex with the auxin analog, leading to degradation of the target protein:
(a) an amino acid sequence in which an amino acid at position 79 of an amino acid sequence set forth in SEQ ID NO: 1 is glycine,
(b) an amino acid sequence in which one to several amino acids are deleted, inserted, substituted, or added in a site other than the amino acid at position 79 of (a), and
(c) an amino acid sequence having 80% or more identity in a site other than the amino acid at position 79 of (a).

The number of amino acids deleted, inserted, substituted or added in (b) is preferably 1 to 120, more preferably 1 to 60, still more preferably 1 to 20, particularly preferably 1 to 10, and most preferably 1 to 5.

In order to be functionally identical to a protein consisting of sequences including the amino acid sequence of (a), the protein has 80% or more identity. With regard to such identity, the identity is more preferably 85% or more, still more preferably 90% or more, particularly preferably 95% or more, and most preferably 99% or more.

Examples of the F79G protein of AtTIR1 include a protein consisting of an amino acid sequence represented by SEQ ID NO: 3.

The first nucleic acid encoding the mutant TIR1 family protein may be a DNA having an exon and an intron, or may be cDNA consisting of an exon. The first nucleic acid encoding the mutant TIR1 family protein may be, for example, a full-length sequence in genomic DNA or a full-length sequence in a cDNA. In addition, the first nucleic acid encoding the mutant TIR1 family protein may be a partial sequence in genomic DNA or a partial sequence in a cDNA as long as an expressed protein functions as TIR1.

In the present specification, the phrase “functioning as the TIR1 family protein” means that, for example, the expressed protein recognizes the degradation tag (the full-length or partial protein of the Aux/IAA family protein) in the presence of the auxin analog. This is because the TIR1 family protein is capable of degrading a target protein labeled with the degradation tag as long as it is capable of recognizing the degradation tag.

In the kit of the present invention, it is preferable that a promoter sequence that controls the transcription of the first nucleic acid be operably linked to the 5′ end of the first nucleic acid encoding the TIR1 family protein. This makes it possible for the TIR1 family protein to be more reliably expressed.

In the present specification, the phrase “being operably linked” means a functional linkage between a gene expression control sequence (for example, a promoter or a series of transcription factor-binding sites) and a gene intended to be expressed (the first nucleic acid encoding the TIR1 family protein). Here, the phrase “expression control sequence” means a sequence that is oriented for the transcription of a gene intended to be expressed (the first nucleic acid encoding the TIR1 family protein).

The promoter is not particularly limited and can be appropriately determined depending on, for example, the type of cell, or the like. Specific examples of the promoter include a CMV promoter, an SV40 promoter, an EFla promoter, and an RSV promoter.

In the kit of the present invention, the first nucleic acid encoding the TIR1 family protein and the promoter sequence operably linked upstream may be in a form of being inserted into a vector.

The vector is preferably an expression vector. The expression vector is not particularly limited and an expression vector suitable for the host cell can be used.

In the vector, a polyadenylation signal, an NLS, a marker gene of a fluorescent protein, or the like may be operably linked to the 5′ end or the 3′ end of the first nucleic acid encoding the TIR1 family protein.

The kit of the present invention may include a non-plant-derived eukaryotic cell or non-human animal having the first nucleic acid on a chromosome.

Such a cell preferably has the first nucleic acid at a safe harbor site.

The phrase “safe harbor site” means a gene region where constant and stable expression occurs, in which a gene that is intrinsically encoded to this region can be kept alive even in the case of being defective or altered. In a case where a foreign DNA (a gene encoding TIR1 in the present embodiment) is inserted into a safe harbor site using a CRISPR system, a PAM sequence is preferably present in the vicinity thereof.

Examples of the safe harbor site include a GTP-binding protein 10 gene locus, a Rosa26 gene locus, a beta-actin gene locus, and an AAVS1 (the AAV integration site 1) gene locus. Among these, in a case of a human-derived cell, a foreign DNA is preferably inserted into the AAVS1 gene locus.

The cell is not particularly limited as long as it is a non-plant-derived eukaryotic cell, and examples thereof include cells of animals, fungi, protists and the like. Examples of the animals include mammals such as humans, mice, rats and rabbits, fish or amphibians such as zebrafish and Xenopus laevis, and invertebrates such as C. elegans and Drosophila.

In addition, examples of the cells also include established eukaryote-derived cells, ES cells, and iPS cells. Specific examples of the eukaryotic cells include established human-derived cells, established mouse-derived cells, established chicken-derived cells, human ES cells, mouse ES cells, human iPS cells, and mouse iPS cells. Specific examples thereof include human HCT116 cells, human HT1080 cells, human NALM6 cells, human ES cells, human iPS cells, mouse ES cells, mouse iPS cells, and chicken DT40 cells.

In addition, examples of the fungi include Saccharomyces cerevisiae and a fission yeast.

The non-human animals having the first nucleic acid on a chromosome are genetically modified non-human animals, in which the degradation of a target protein in vivo is controlled by an auxin-degron system. Examples of the non-human animals include cats, dogs, horses, monkeys, cows, sheep, pigs, goats, rabbits, hamsters, guinea pigs, rats, and mice. Among these, rodents are preferable from the viewpoint of their record in drug evaluation. Examples of the rodents include hamsters, guinea pigs, rats, and mice, and rats and mice are preferable.

### <Auxin Analog>

In the present invention, the auxin analog is a compound represented by General Formula (I).

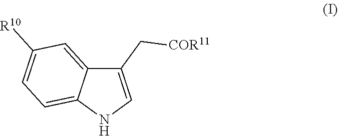

(In General Formula (I), R¹⁰ is a cyclic aliphatic hydrocarbon group in which a substituent may be present and some of the carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

### [Cyclic Aliphatic Hydrocarbon Group]

The cyclic aliphatic hydrocarbon group as R¹⁰ may be a monocyclic group or a polycyclic group. Examples of the monocyclic aliphatic hydrocarbon include a cyclopropyl group, a cyclobutyl group, a cyclopentyl group, a cyclohexyl group, a methylcyclohexyl group, a dimethylcyclohexyl group, a cycloheptyl group, a cyclooctyl group, a cyclononyl group, and a cyclodecyl group.

Examples of the polycyclic alicyclic hydrocarbon group include a decahydronaphthyl group, an adamantyl group, a 2-alkyladamantan-2-yl group, a 1-(adamantan-1-yl)alkan-1-yl group, a norbornyl group, a methylnorbornyl group, and an isobornyl group.

In the cyclic aliphatic hydrocarbon group, some of the carbon atoms constituting a ring may be substituted with heteroatoms. Examples of the heteroatom include an oxygen atom, a sulfur atom, and a nitrogen atom. Examples of such a heterocyclic ring include pyrrolidine, tetrahydrofuran, tetrahydrothiophene, piperidine, tetrahydropyran, tetrahydrothiopyran, dioxane, and dioxolane.

Examples of the substituent include an alkyl group having 1 to 6 carbon atoms, an alkoxy group having 1 to 6 carbon atoms, a halogen atom, and an aryl group having 6 to 30 carbon atoms.

Examples of the alkyl group include a methyl group, an ethyl group, an n-propyl group, an isopropyl group, a cyclopropyl group, an n-butyl group, an isobutyl group, a sec-butyl group, a tert-butyl group, a cyclobutyl group, an n-pentyl group, an isopentyl group, a sec-pentyl group, a neopentyl group, a tert-pentyl group, a cyclopentyl group, a 2,3-dimethylpropyl group, a 1-ethylpropyl group, a 1-methylbutyl group, a 2-methylbutyl group, an n-hexyl group, an isohexyl group, a cyclohexyl group, a 2-methylpentyl group, a 3-methylpentyl group, a 1,1,2-trimethylpropyl group, and a 3,3-dimethylbutyl group.

Examples of the alkoxy group include an alkoxy group in which the R portion of —OR is the same as that of the above-described alkyl group having 1 to 6 carbon atoms. Among those, as the alkoxy group having carbon atoms, a methoxy group or an ethoxy group is preferable.

Examples of the halogen atom include a fluorine atom, a chlorine atom, a bromine atom, and an iodine atom. Among these, the fluorine atom or the chlorine atom is preferable as the halogen atom.

Examples of the aryl group include a phenyl group, a naphthyl group, a benzyl group, a phenethyl group, a biphenyl group, a pentalenyl group, an indenyl group, an anthranyl group, a tetracenyl group, a pentacenyl group, a pyrenyl group, a peryleneyl group, a fluorenyl group, and a phenanthryl group.

### [Aromatic Hydrocarbon Group]

Examples of the aromatic hydrocarbon group as R¹⁰ include the above-described aryl group having 6 to 30 carbon atoms.

In the aromatic hydrocarbon group, some of the carbon atoms constituting a ring may be substituted with heteroatoms. Examples of the heteroatom include an oxygen atom, a sulfur atom, and a nitrogen atom. Examples of such a heterocyclic ring include pyrrole, furan, thiophene, pyridine, imidazole, pyrazole, oxazole, thiazole, pyridazine, pyrimidine, indole, benzimidazole, quinoline, isoquinoline, chromene, and isochromene.

Examples of the substituent include the same substituents as those mentioned in the section of [Cyclic Aliphatic Hydrocarbon Group].

Among the compounds represented by General Formula (I), a compound represented by General Formula (I-1) is preferable as the compound in which R¹⁰ is an aromatic hydrocarbon group.

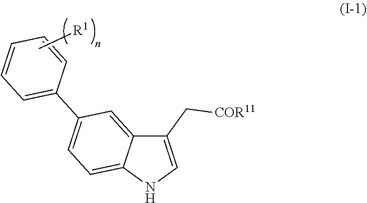

(In General Formula (I-1), R¹ is an alkyl group having 1 to 6 carbon atoms, an alkoxy group having 1 to 6 carbon atoms, a halogen atom, or an aryl group having 6 to 30 carbon atoms. n is an integer of 0 to 5, and in a case where n is an integer of 2 to 5, n pieces of R¹'s may be the same as or different from each other. R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

Examples of the halogen atom of R¹ include a fluorine atom, a chlorine atom, a bromine atom, and an iodine atom. Among these, the fluorine atom or the chlorine atom is preferable as the halogen atom.

Examples of the alkyl group of R¹ include a methyl group, an ethyl group, an n-propyl group, an isopropyl group, a cyclopropyl group, an n-butyl group, an isobutyl group, a sec-butyl group, a tert-butyl group, a cyclobutyl group, an n-pentyl group, an isopentyl group, a sec-pentyl group, a neopentyl group, a tert-pentyl group, a cyclopentyl group, a 2,3-dimethylpropyl group, a 1-ethylpropyl group, a 1-methylbutyl group, a 2-methylbutyl group, an n-hexyl group, an isohexyl group, a cyclohexyl group, a 2-methylpentyl group, a 3-methylpentyl group, a 1,1,2-trimethylpropyl group, and a 3,3-dimethylbutyl group. Among these, the methyl group or the ethyl group is preferable as the alkyl group having 1 to 6 carbon atoms.

Examples of the alkoxy group of R¹ include those of the alkoxy groups in which the R portion of —OR is the same as that of the above-described alkyl group having 1 to 6 carbon atoms. Among those, as the alkoxy group having carbon atoms, a methoxy group or an ethoxy group is preferable.

Examples of the aryl group of R¹ include a phenyl group, a naphthyl group, a benzyl group, a phenethyl group, a biphenyl group, a pentalenyl group, an indenyl group, an anthranyl group, a tetracenyl group, a pentacenyl group, a pyrenyl group, a peryleneyl group, a fluorenyl group, and a phenanthryl group.

n of R¹ is an integer of 0 to 5, and preferably 0 to 3. Examples of the compound represented by General Formula (I-1) include the following compounds.

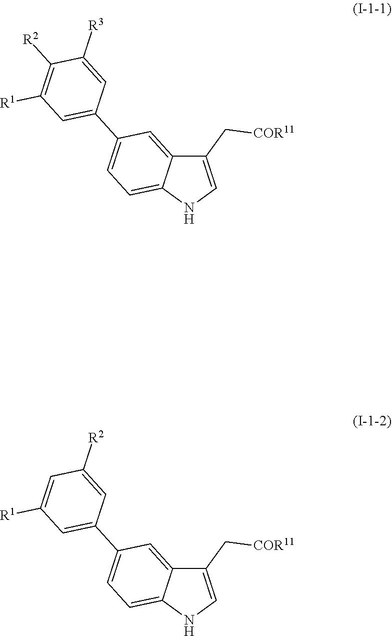

(In General Formulae (I-1-1) and (I-1-2), R¹ to R³ are each independently an alkyl group having 1 to 6 carbon atoms, an alkoxy group having 1 to 6 carbon atoms, a halogen atom, or an aryl group having 6 to 30 carbon atoms. R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

In General Formulae (I-1-1) and (I-1-2), R¹ to R³ are the same as R¹ in General Formula (I-1).

Examples of the compound represented by General Formula (I-1) include the following compounds.

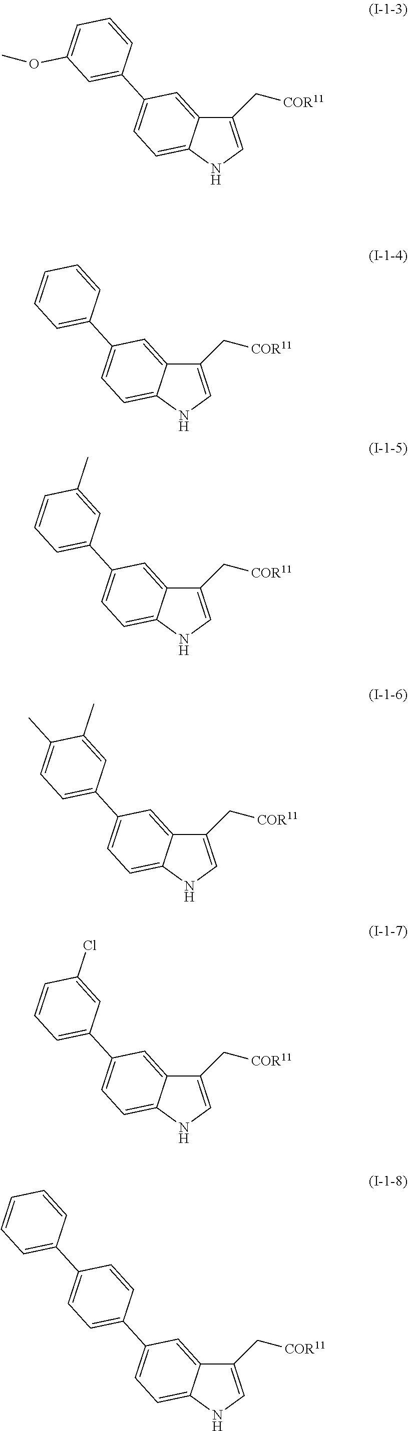

(In General Formulae (I-1-3) to (I-1-8), R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

In addition, examples of the compound in which R¹⁰ is an aromatic hydrocarbon group include compounds represented by the following formulae (I-2) to (I-5).

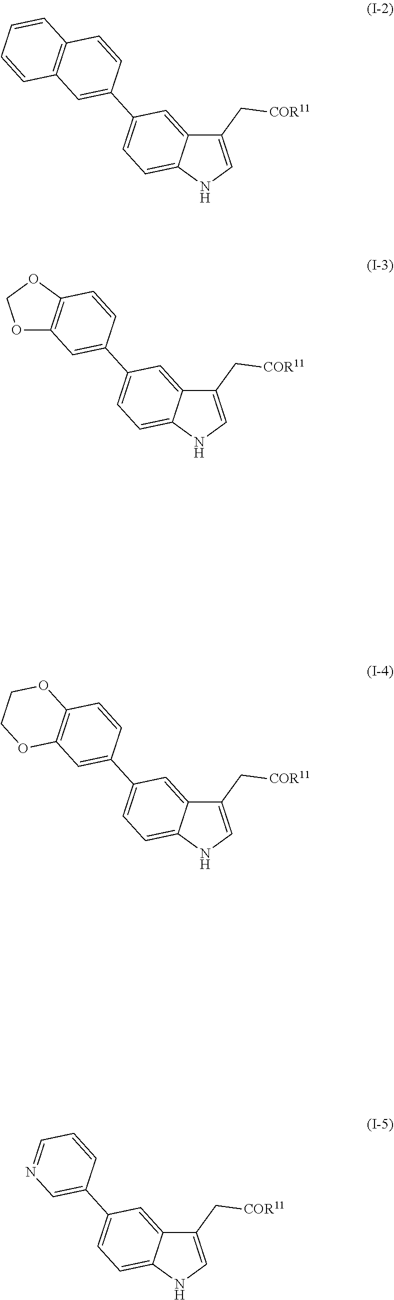

(In General Formulae (I-2) to (I-5), R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

In addition, examples of the compound in which R¹⁰ is a cyclic aliphatic hydrocarbon group include a compound represented by General Formula (I-6).

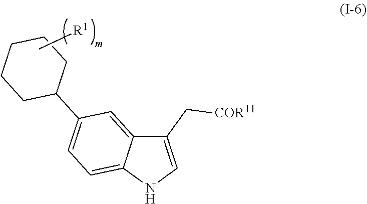

(In General Formula (I-6), R¹ is an alkyl group having 1 to 6 carbon atoms, an alkoxy group having 1 to 6 carbon atoms, a halogen atom, or an aryl group having 6 to 30 carbon atoms. m is an integer of 0 to 11, and in a case where m is an integer of 2 to 11, m pieces of R¹'s may be the same as or different from each other. R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

R¹ is the same as that mentioned in General Formula (I-1). m is preferably 0 to 6, and more preferably 0 to 3.

In addition, examples of the compound in which R¹⁰ is a cyclic aliphatic hydrocarbon group include a compound represented by Formula (I-7).

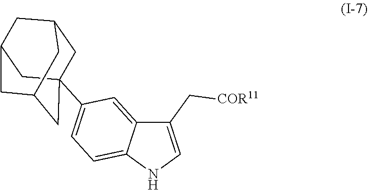

(In General Formula (I-7), R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and the plurality of R¹³'s are each independently a hydrogen atom or an alkyl group having 1 to 6 carbon atoms.)

R¹¹ is a substituent that contributes to membrane permeability, and is —O—(CH₂)ₙ₁—O—C(═O)R¹², —OR¹², or —N(R¹³)₂.

In —O—(CH₂)ₙ₁—O—C(═O)R¹², R¹² is an alkyl group having 1 to 6 carbon atoms, and examples thereof include the same groups as those of the alkyl group of R¹, among which the methyl group or the ethyl group is preferable.

In —(CH₂)ₘ—O—C(═O)R¹², n1 is an integer of 1 to 6, and preferably 1.

In a case where R¹¹ is —O—(CH₂)ₙ₁—O—C(═O)R¹², the following compounds are preferable.

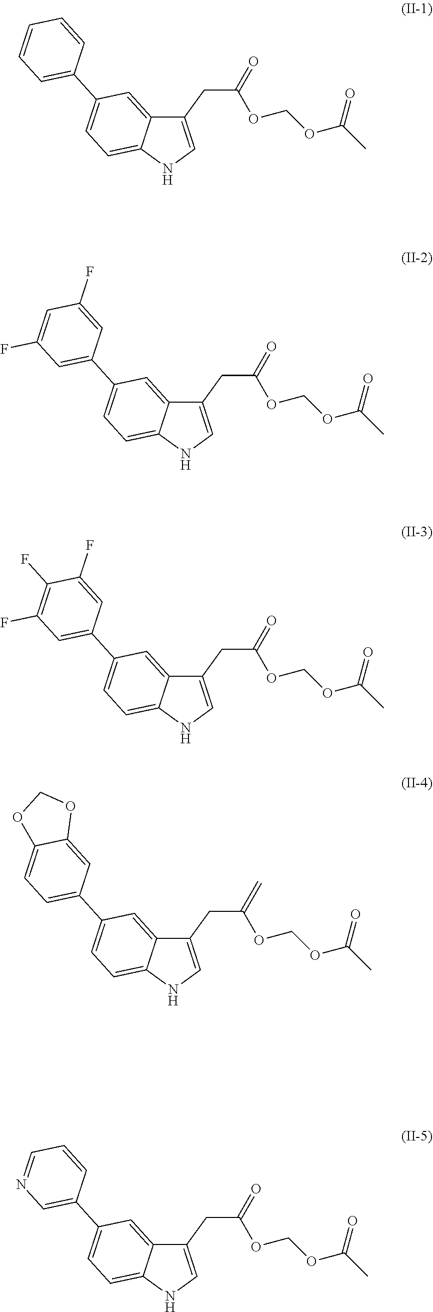 [SMILES: CC(=O)OCOC(=O)Cc1c[nH]c2ccc(-c3ccccc3)cc12 ; CC(=O)OCOC(=O)Cc1c[nH]c2ccc(-c3cc(F)cc(F)c3)cc12 ; CC(=O)OCOC(=O)Cc1c[nH]c2ccc(-c3cc(F)c(F)c(F)c3)cc12 ; C=C(Cc1c[nH]c2ccc(-c3ccc4c(c3)OCO4)cc12)OCOC(C)=O ; CC(=O)OCOC(=O)Cc1c[nH]c2ccc(-c3cccnc3)cc12]

In a case where R¹¹ is —OR¹², R¹² is an alkyl group having 1 to 6 carbon atoms, and examples thereof include the same groups as those of the alkyl group of R¹, among which the methyl group or the ethyl group is preferable.

In a case where R¹¹ is —OR¹², the following compounds are preferable.

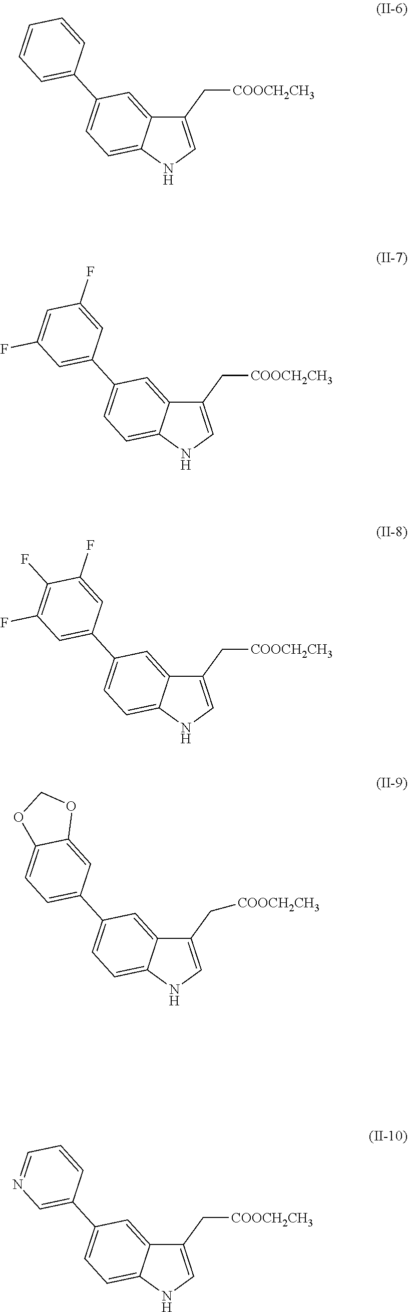

In a case where R¹¹ is —N(R¹³)₂, a plurality of R¹³'s are each independently a hydrogen atom or an alkyl group having 1 to 6 carbon atoms. Examples of the alkyl group of R¹³ include the same groups as those of the alkyl group of R¹, among which the methyl group or the ethyl group is preferable.

### [Other Compounds]

Examples of other compounds include compounds represented by the following general formulae.

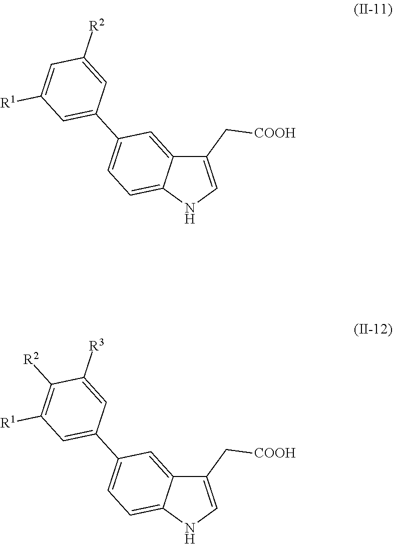

(In General Formulae (II-11) and (II-12), R¹ to R³ are each independently an alkyl group having 1 to 6 carbon atoms, an alkoxy group having 1 to 6 carbon atoms, a halogen atom, or an aryl group having 6 to 30 carbon atoms.

Among those, the following compounds are preferable.

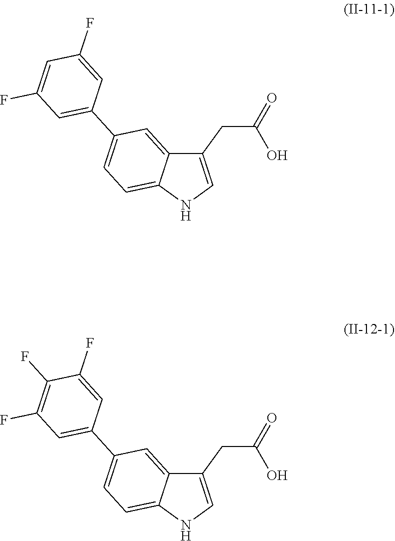 [SMILES: O=C(O)Cc1c[nH]c2ccc(-c3cc(F)cc(F)c3)cc12 ; O=C(O)Cc1c[nH]c2ccc(-c3cc(F)c(F)c(F)c3)cc12]

In addition, examples of other compounds include compounds represented by the following general formulae.

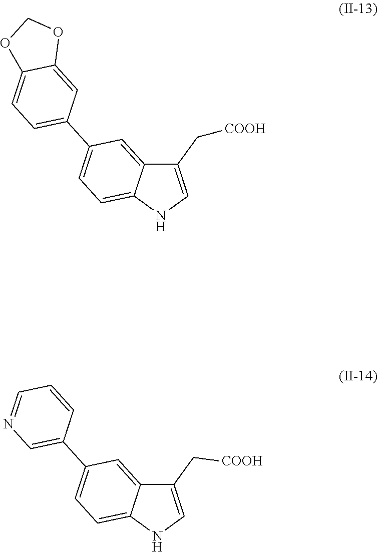

### <Second Nucleic Acid>

In the present invention, the second nucleic acid encodes a degradation tag that includes at least a partial Aux/IAA family protein and has an affinity for a complex of the mutant TIR1 family protein and the auxin analog.

The type of gene encoding the Aux/IAA family protein is not particularly limited as long as the gene is a plant-derived Aux/IAA family gene. Specific examples of the gene encoding the Aux/IAA family protein include an IAA1 gene, an IAA2 gene, an IAA3 gene, an IAA4 gene, an IAA5 gene, an IAA6 gene, an IAA7 gene, an IAA8 gene, an IAA9 gene, an IAA10 gene, an IAA11 gene, an IAA12 gene, an IAA13 gene, an IAA14 gene, an IAA15 gene, an IAA16 gene, an IAA17 gene, an IAA18 gene, an IAA19 gene, an IAA20 gene, an IAA26 gene, an IAA27 gene, an IAA28 gene, an IAA29 gene, an IAA30 gene, an IAA31 gene, an IAA32 gene, an IAA33 gene, and an IAA 34 gene.

The kit of the present invention may have any one type of full-length or partial sequence of the gene encoding the Aux/IAA family protein, or may have two or more types thereof. For example, the sequence of an Arabidopsis thaliana-derived Aux/IAA family gene is registered in the Arabidopsis Information Resource (TAIR), and the accession number of each gene is as follows.

The IAA1 gene (AT4G14560), the IAA2 gene (AT3G23030), the IAA3 gene (AT1G04240), the IAA4 gene (AT5G43700), the IAA5 gene (AT1G15580), the IAA6 gene (AT1G52830), the IAA7 gene (AT3G23050), the IAA8 gene (AT2G22670), the IAA9 gene (AT5G65670), the IAA10 gene (AT1G04100), the IAA11 gene (AT4G28640), the IAA12 gene (AT1G04550), the IAA13 gene (AT2G33310), the IAA14 gene (AT4G14550), the IAA15 gene (AT1G80390), the IAA16 gene (AT3G04730), the IAA17 gene (AT1G04250), the IAA18 gene (AT1G51950), the IAA19 gene (AT3G15540), the IAA20 gene (AT2G46990), the IAA26 gene (AT3G16500), the IAA27 gene (AT4G29080), the IAA28 gene (AT5G25890), the IAA29 gene (AT4G32280), the IAA30 gene (AT3G62100), the IAA31 gene (AT3G17600), the IAA32 gene (AT2G01200), the IAA33 gene (AT5G57420), and the IAA34 gene (AT1G15050).

Among these, the Arabidopsis thaliana IAA17 gene is preferable.

The degradation tag is not particularly limited as long as it is bound with the complex of the mutant TIR1 family protein and the auxin analog, leading to degradation of the target protein, and the degradation tag preferably includes a full-length or partial protein of the mAID or a full-length or partial protein of the AID* among the Aux/IAA family proteins.

“mAID” is the abbreviation for “mini-auxin-inducible degron” and is a protein consisting of a partial sequence of the Arabidopsis thaliana IAA17, which is one of the Aux/IAA family proteins. This partial sequence is a sequence consisting of a region including at least two Lys residues on each of the N-terminal side and the C-terminal side of a domain II region of the Aux/IAA family protein or a sequence formed by linking two or more sequences described above. This mAID can serve as a degradation tag that labels the target protein. For example, the amino acid sequence of the mAID is represented by SEQ ID NO: 4.

“AID” is a protein that consists of an IAA17-derived sequence having a length slightly different from that of the mAID, and consists of a short amino acid sequence of 44aa. For example, the amino acid sequence of the AID* is represented by SEQ ID NO: 5.

### <Third Nucleic Acid>

In a case where the target protein has been determined, the kit of the present invention may include a third nucleic acid encoding the target protein, which is linked upstream or downstream of the second nucleic acid. The second nucleic acid may be arranged to be adjacent to either the 5′ side or the 3′ side of the third nucleic acid.

A fused nucleic acid consisting of the second nucleic acid and the third nucleic acid preferably has a promoter sequence operably linked thereto in the same manner as with the first nucleic acid, and may be combined into an expression vector.

### <<Method for Degrading Target Protein>>

The method for degrading a target protein of the present invention is a method using the above-described kit of the present invention. The use of the above-described kit in the auxin-degron system makes it possible to control the degradation of a target protein with high efficiency.

Examples of the method of controlling the degradation of a target protein using the above-described kit include the following methods.

First, a target protein labeled with a degradation tag and a TIR1 family protein are expressed in a cell. The target protein labeled with the degradation tag and the TIR1 family protein are preferably expressed constantly.

Next, an auxin analog is added to a culture medium. The concentration of the auxin analog included in the culture medium is not limited, and is, for example, 1 μM or more and less than 0.1 mM, and preferably 10 nM or more and 100 μM or less. As will be described later in Examples, the combination of 5-Ph-IAA-AM and AtTIR1 (F79G) can efficiently induce the degradation of a target protein. A predetermined concentration of the auxin analog is added and thus incorporated into the cell, and then R¹¹ is degraded and becomes hydrophilic to form a complex of the mutant TIR1 family protein and the auxin analog. This complex recognizes the target protein labeled with the degradation tag, and the degradation of the target protein is induced.

In addition, in a case where a non-human animal is used, the target protein is degraded by administering an auxin analog.

A method for administrating the auxin analog is not particularly limited, and examples thereof include intraperitoneal administration, intravenous administration, intraarterial administration, intramuscular administration, intradermal administration, subcutaneous administration, and oral administration. The administration amount is, for example, preferably 0.1 mg/kg to 100 mg/kg, more preferably 0.2 mg/kg to 50 mg/kg, and still more preferably 0.5 mg/kg to 20 mg/kg per day. The administration period is preferably 1 to 10 days, and more preferably 3 to 7 days.

### [Inhibitor]

In addition, a TIR1 auxin receptor antagonist may be used in order to further control the basal degradation of the target protein in a case where no ligand is added.

Examples of the TIR1 auxin receptor antagonist include a compound represented by the following general formula.

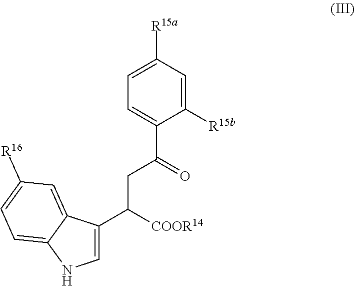

(In General Formula (III), R¹⁴ is a hydrogen atom or an alkyl group having 1 to 6 carbon atoms. R^{15a} and R1^{5b} are each independently a hydrogen atom, a halogen atom, or an alkyl group having 1 to 6 carbon atoms. R¹⁶ is a hydrogen atom, an alkyl group having 1 to 6 carbon atoms, an alkoxy group having 1 to 6 carbon atoms, a cyclic aliphatic hydrocarbon group in which a substituent may be present and some of the carbon atoms constituting a ring may be substituted with heteroatoms, or an aromatic hydrocarbon group in which a substituent may be present and some of the carbon atoms constituting a ring may be substituted with heteroatoms.)

Examples of the alkyl group of R¹⁴, R^{15a}, R^{15b}, and R¹⁶ include a methyl group, an ethyl group, an n-propyl group, an isopropyl group, a cyclopropyl group, an n-butyl group, an isobutyl group, a sec-butyl group, a tert-butyl group, a cyclobutyl group, an n-pentyl group, an isopentyl group, a sec-pentyl group, a neopentyl group, a tert-pentyl group, a cyclopentyl group, a 2,3-dimethylpropyl group, a 1-ethylpropyl group, a 1-methylbutyl group, a 2-methylbutyl group, an n-hexyl group, an isohexyl group, a cyclohexyl group, a 2-methylpentyl group, a 3-methylpentyl group, a 1,1,2-trimethylpropyl group, and a 3,3-dimethylbutyl group.

Examples of the cyclic aliphatic hydrocarbon group of R¹⁶, in which a substituent may be present and some of the carbon atoms constituting a ring may be substituted with heteroatoms, include the same groups as those of R¹⁰, and the cyclic aliphatic hydrocarbon group is preferably a monocyclic or polycyclic aliphatic hydrocarbon group, more preferably a cycloalkyl group having 1 to 10 carbon atoms or an adamantyl group, and particularly preferably a cyclohexyl group or an adamantyl group.

Examples of the aromatic hydrocarbon group of R¹⁶, in which a substituent may be present and some of the carbon atoms constituting a ring may be substituted with heteroatoms, include the same groups as those of R¹⁰, and the aromatic hydrocarbon group is preferably an aryl group having 6 to 30 carbon atoms, and more preferably a phenyl group.

Examples of the preferred TIR1 auxin receptor antagonist include a compound represented by the following general formula.

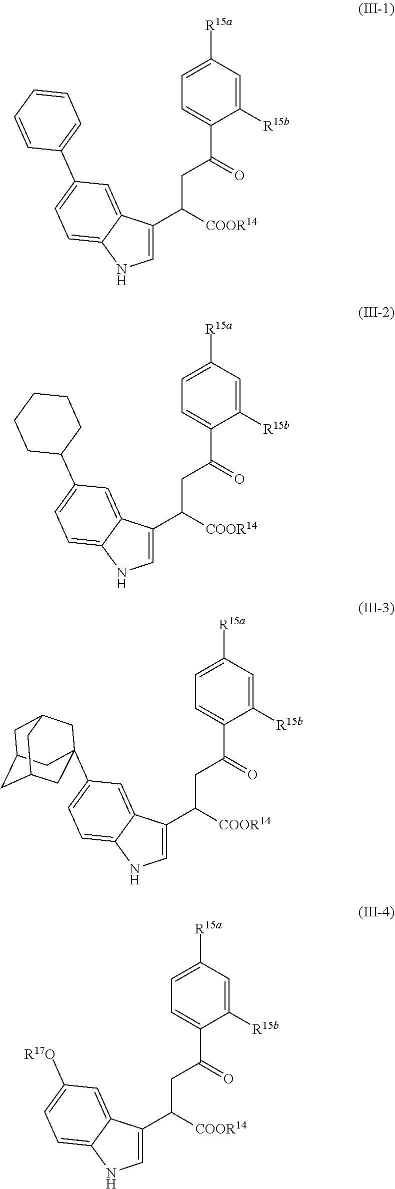

(In General Formulae (III-1) and (III-2), R¹⁴ is a hydrogen atom or an alkyl group having 1 to 6 carbon atoms. R^{15a} and R1^{Sb} are each independently a hydrogen atom, a halogen atom, or an alkyl group having 1 to 6 carbon atoms. R¹⁷ is an alkyl group having 1 to 6 carbon atoms.)

Examples of the alkyl group of R¹⁷ include the same groups as those of the alkyl group of R¹⁶.

Examples of the preferred TIR1 auxin receptor antagonist include a compound represented by the following formula.

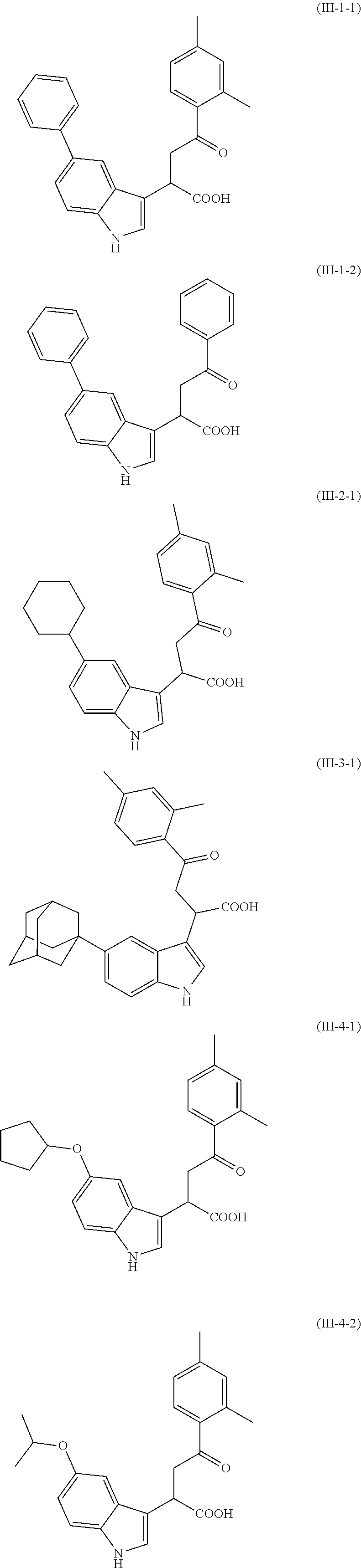

The TIR1 auxin receptor antagonist is required to have an ability to bind to a TIR1 family protein, but does not have an ability to bind to a degradation-inducing peptide (in particular, an mAID). As a result, the binding of the TIR1 family protein to the degradation-inducing peptide (in particular, an mAID) can be inhibited.

Examples of the degradation method of the present invention include, in a case of the method using the TIR1 auxin receptor antagonist, the following method.

First, in a eukaryotic cell, a target protein and a TIR1 family protein labeled with a degradation-inducing peptide are expressed in the presence of a TIR1 auxin receptor antagonist. This makes it possible to suppress the basal degradation of the target protein.

Next, a culture medium including the TIR1 auxin receptor antagonist is replaced with a culture medium including the above-described auxin analog. As a result, the TIR1 auxin receptor antagonist is removed and the auxin analog is present instead, whereby the target protein labeled with the degradation-inducing peptide is degraded by the TIR1 family protein.

According to the degradation method of the present invention, it is possible to induce the degradation of a target protein in an auxin analog-specific manner.

### <<Compound>>

The compound of the present invention is a compound represented by General Formula (I).

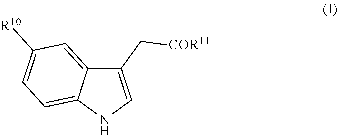

(In General Formula (I), R¹⁰ is a cyclic aliphatic hydrocarbon group in which a substituent may be present and some of the carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms.)

In a case where R¹¹ is —O—(CH₂)ₙ₁—O—C(═O)R¹² in General Formula (I), the following synthesis method can be adopted.

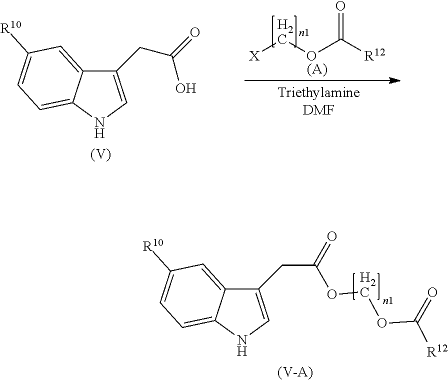

(In General Formulae (V) and (V-A), R¹⁰ is a cyclic aliphatic hydrocarbon group in which a substituent may be present and some of the carbon atoms constituting a ring may be substituted with heteroatoms, or an aromatic hydrocarbon group in which a substituent may be present and some of the carbon atoms constituting a ring may be substituted with heteroatom. In General Formula (A), X is a halogen. In General Formulae (A) and (V-A), n1 is an integer of 1 to 6, and R¹² is an alkyl group having 1 to 6 carbon atoms.)

The compound represented by General Formula (A) is added dropwise to a solution of the compound represented by General Formula (V) and trimethylamine in DMF, and the mixture is stirred at room temperature. The reaction mixture is added to water, the mixture is extracted with EtOAc, the organic layer is washed and dried, and the residue is purified to obtain a compound represented by General Formula (V-A).

In General Formula (A), examples of the halogen atom include a fluorine atom, a chlorine atom, a bromine atom, and an iodine atom.

In the compound represented by General Formula (V-A), preferred examples of the compound include the same compounds as those mentioned in <Auxin Analog>.

### EXAMPLES

The present invention will be described below with reference to Examples, but is not limited to the following Examples.

### Example 1

### [Synthesis of 5-Ph-IAA]

A compound represented by Formula (VI) (also referred to as 5-Ph-IAA) was synthesized.

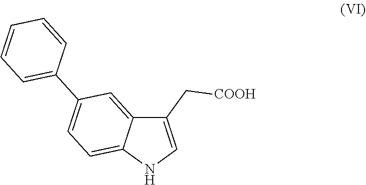 [SMILES: O=C(O)Cc1c[nH]c2ccc(-c3ccccc3)cc12]

[Construction of AID2 System in Nematodes (C. elegans)]

As described below, the degradation system according to the present invention (also referred to as the AID2 system) was constructed.

An F79G mutation was introduced into an AtTIR1 gene of nematodes, including mutations of D170E and M473L for improving an affinity for an auxin. Furthermore, the F79G mutation corresponds to F74G in OsTLR1. A gene encoding a fusion protein (AtTIR1 (F79G)::mRuby) of the AtTIR1 (F79G) and a red fluorescent protein was inserted into the ttTi5605 locus (chromosome II: position 8, 420, and 204) by a CRISPR-Cas9-mediated knock-in.

The expression of AtTIR1 (F79G)::mRuby was driven by an eft3277 promoter to achieve ubiquitous expression in all somatic cells.

A lineage expressing AtTIR1 (F79G)::mRuby was crossed with a lineage expressing AID* (see SEQ ID NO: 5) (AID*::GFP) fused with a GFP reporter.

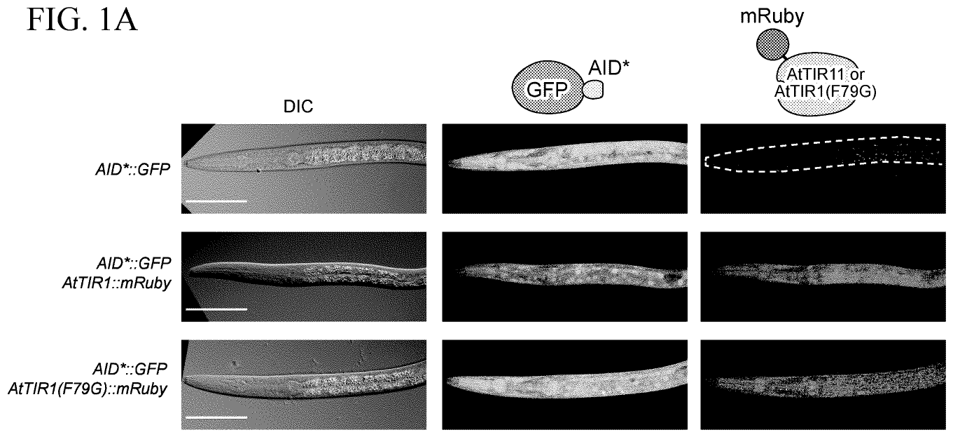

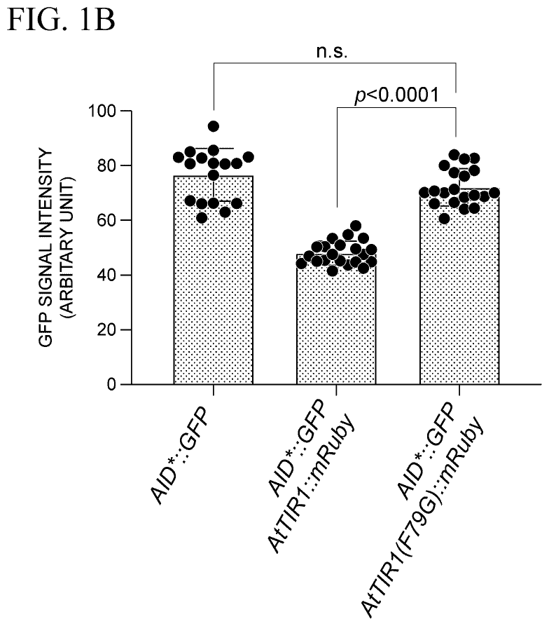

In order to monitor the levels of basal degradation, the intensity of AID*::GFP in larvae expressing AtTIR1::mRuby or AtTIR1 (F79G)::mRuby was measured. In the strain expressing AtTIR1::mRuby, the reporter intensity was reduced to 73%, as compared to the parent strain without the transgene, and the basal degradation in a case of no addition of a ligand was shown (see FIGS. 1A and 1B). In contrast, the intensity of the reporter was not affected at all in the larvae expressing AtTIR1 (F79G)::mRuby (see FIGS. 1A and 1B). This indicated that the basal degradation of AtTIR1 (F79G) was at a low level in nematodes.

Next, in order to confirm that 5-Ph-IAA is safer than IAA against nematodes, L1 larvae expressing AID*::GFP were each grown with AtTIR1 or AtTIR1 (F79G) in the presence of 1 mM IAA or 5 μM 5-Ph-IAA.

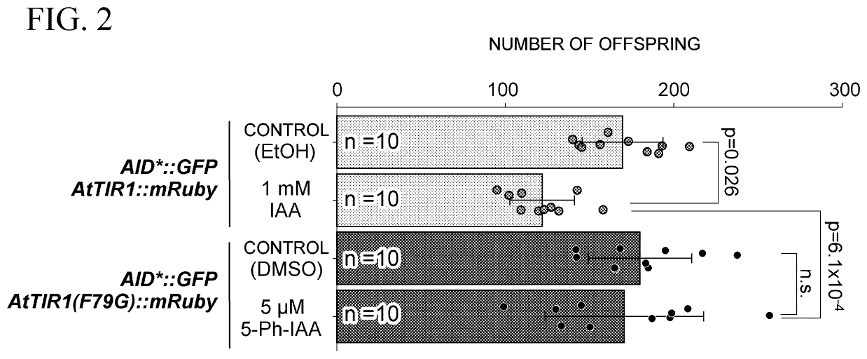

As shown in FIG. 2, it was confirmed that the number of progeny was reduced in the nematodes treated with 1 mM IAA. In contrast, the nematodes treated with 5 μM 5-Ph-IAA showed no difference. From this result, it was confirmed that the treatment of the nematodes with 5 μM 5-Ph-IAA is safer than the treatment of the nematodes with 1 mM IAA.

In order to test whether the combination of AtTIR1 (F79G) and 5-Ph-IAA can induce the degradation of the AID*::GFP reporter, the larvae including AtTIR1 or AtTIR1 (F79G) were each treated on a plate including 1 mM IAA or 5 μM 5PhIAA.

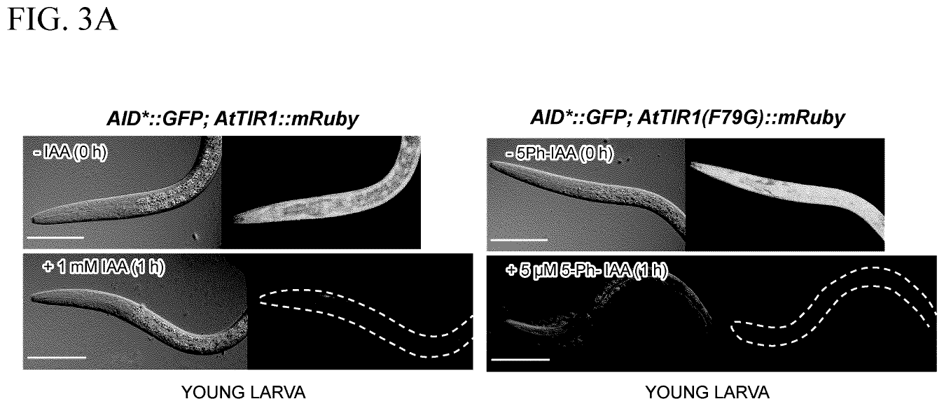

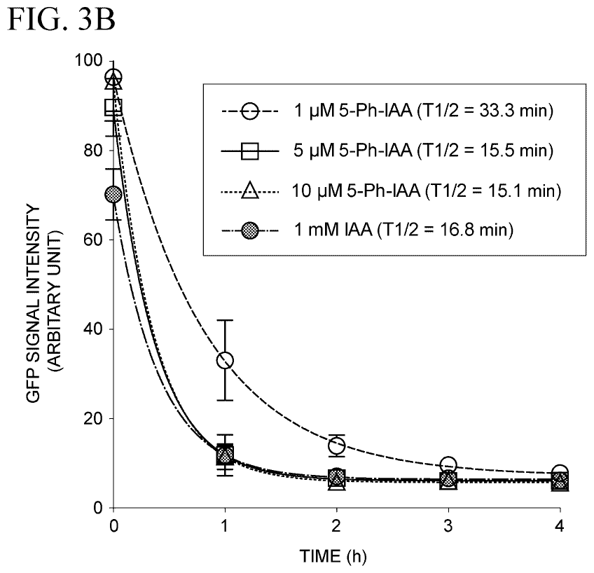

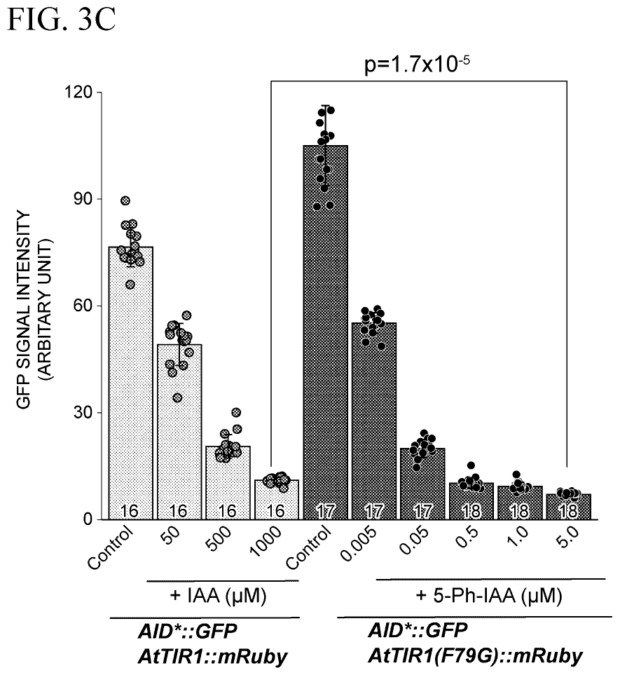

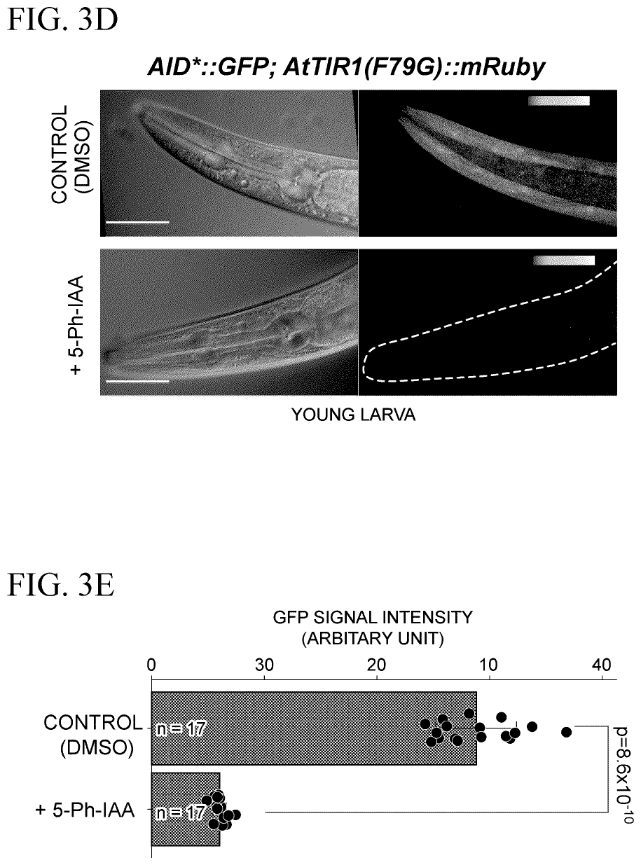

Before the treatment, the fluorescence intensity of the reporter was higher in the larvae expressing AtTIR1 (F79G) than in the larvae expressing AtTIR1 (FIGS. 3A and 3B). The treatment with 1 mM IAA reduced the level of the reporter in the larvae expressing AtTIR1 with a half-life of 16.8 min. The treatment with 5 μM 5-Ph-IAA resulted in a similar decrease in reporter level with a half-life of 15.5 min in the larvae expressing AtTIR1 (F79G) despite a 500-fold lower ligand concentration (see FIGS. 3A and 3B). Furthermore, as a result of the treatment with 1 μM 5-Ph-IAA and 10 μM 5-Ph-IAA, the half-lives were each 33.6 min and 15.1 min (see FIG. 3B).

These results showed that the removal rate reaches almost the maximum in a case of the treatment with 5 μM 5-Ph-IAA. Due to the low level of basal degradation, sharper degradation could be achieved with the combination of AtTIR1 (F79G) and 5 μM 5-Ph-IAA, as compared with the AID system equipped with AtTIR1 and 1 mM IAA in the related art.

Next, L1 larvae were grown on plates including ligands at different concentrations. The intensity of the AID*::GFP reporter of larvae (second generation) born from adults was monitored. The lowest reporter expression was observed in the larvae treated with 5.0 μM 5-Ph-IAA (see FIG. 3C). Furthermore, this expression level was lower than the expression level of the AtTIR1-expressing larvae treated with 1 mM IAA (p=1.7×10⁻⁵).

The estimated DC50 value (ligand concentrations required for 50% degradation) was 4.5 nM in 5-Ph-IAA combined with AtTIR1 (F79G), and the estimated DC50 value was 59 μM in IAA combined with AtTIR1. This result indicates that the AID2 system using 5-Ph-IAA requires a ligand concentration about 1,300 times lower than that of the AID system in a long-term treatment in the related art. Furthermore, the efficacy of the AID2 system in adults was tested. In a case where the adults were treated with 5 μM 5-Ph-IAA for 4 hours, a loss of the reporter was observed (see FIGS. 3D and 3E). These results clearly showed that the combination of AtTIR1 (F79G) and 5-Ph-IAA improves the ligand sensitivity and more rapidly removes target proteins in the larvae and the adults of nematodes.

### Example 2

[Examination of Effect of 5-pH-IAA in Embryos]

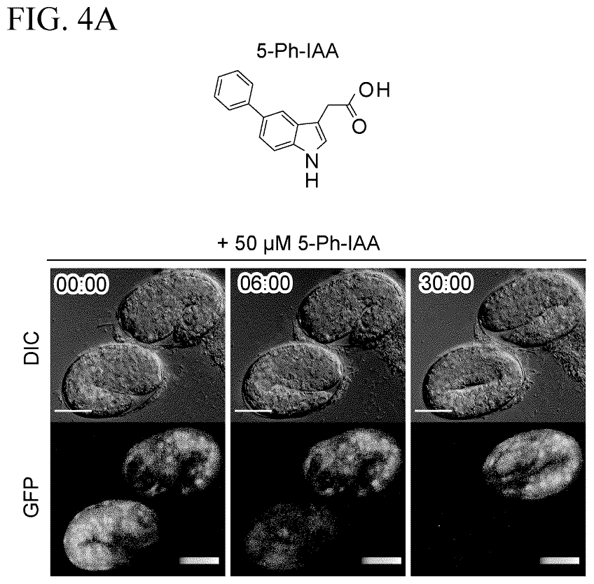

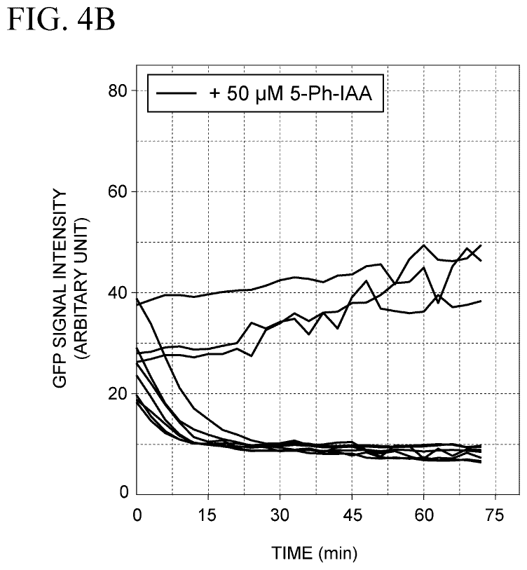

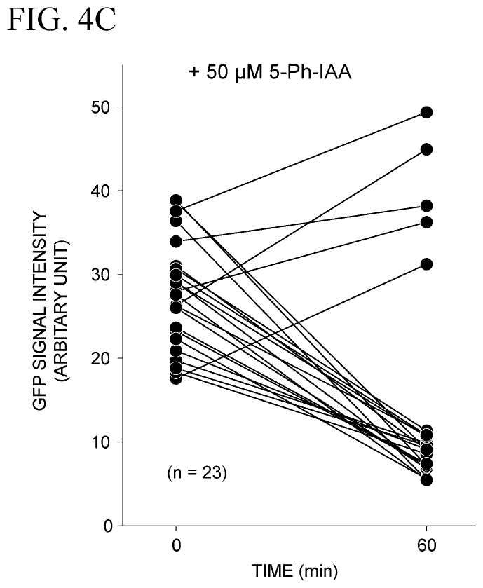

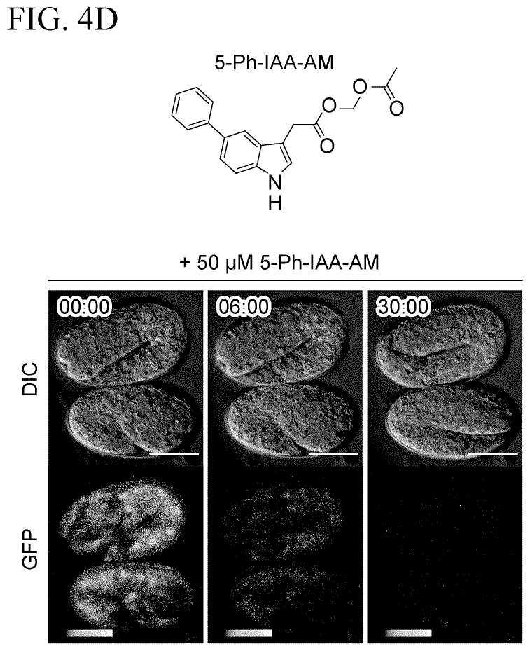

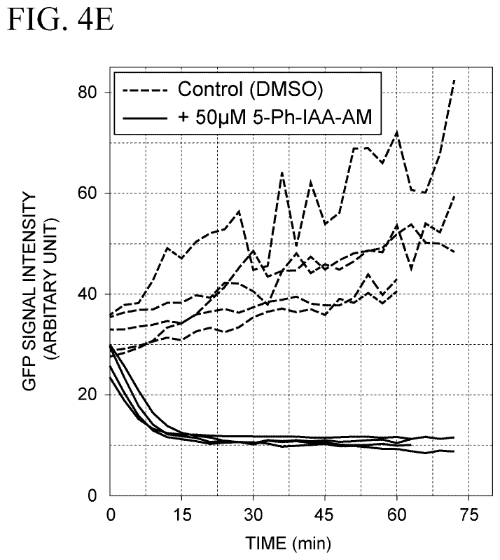

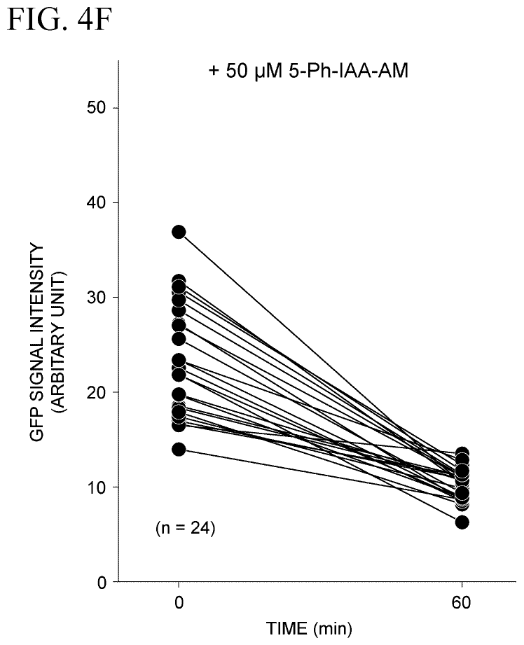

Rapid removal of the target in the embryos is useful for analyzing a role of a protein in embryogenesis. Initially, 5-Ph-IAA was used to test the efficacy of protein removal in embryos. An egg-planting embryo expressing AtTIR1 (F79G)::mRuby and AID*::GFP was placed in a droplet of an M9 buffer including 50 μM 5-Ph-IAA, and then the reporter intensity was monitored (see FIG. 4A).

It was confirmed that in the treatment with 50 μM 5-Ph-IAA, the fluorescence intensity caused a rapid decrease in some embryos (78%, 18/23 embryos) (see FIGS. 4B and 4C). In addition, in a case where 5-Ph-IAA at a higher concentration of 100 μM was tested, almost the same results (80%, 16/20) were observed.

In a case where the degradation was induced by 50 μM 5-Ph-IAA, the degradation of the reporter reached the maximum within 15 to 30 min (see FIGS. 4A and 4B). However, there were some embryos that did not react to the treatments (see FIG. 4A, upper embryo). It is considered that those results suggest that the embryos of C. elegans develop within the eggshell, and the eggshell blocks the permeability of many compounds.

In order to improve the permeability into the eggshell, 5-Ph-IAA-AM having an acetoxymethyl group was synthesized (see FIG. 4D).

### [Synthesis of 5-Ph-IAA AM Ester]

For ¹H and ¹³C NMR spectra, (JEOL, Japan) chemical shifts recorded with a JEOL ECZ400 NMR spectrometer are shown as δ values from TMS as an internal reference. The peak multiplicities are expressed in Hz. Column chromatography was performed using Merck silica gel 60 (230 to 400 mesh, Merck, Japan). All chemical substances were purchased from Tokyo Chemical Industry Co., Ltd. (Tokyo, Japan).

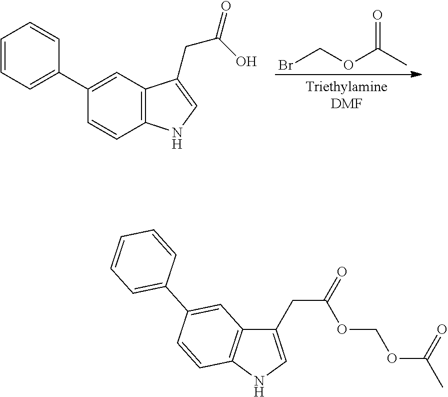

Bromomethyl acetate (300 mg, 2.0 mmol) was added dropwise to a solution of 5-phenylindole-3-acetic acid (500 mg, 2.0 mmol) and trimethylamine (402 mg, 4.0 mmol) in DMF (10 mL), and the mixture was stirred at room temperature for 18 hours.

The reaction mixture was added to water (100 mL) and extracted with EtOAc (50 mL×3). The organic layer was washed with 1 M HCl, 1 M aqueous Na₂CO₃, and brine, and then dried over Na₂SO₄. The residue was purified by a silica gel column chromatography (hexane:EtOAc=7:3) to obtain a 5-phenylindole 3-acetic acid acetoxymethyl ester as a crystal (442 mg, yield: 69%).

The analysis results of ¹H-NMR and ¹³C-NMR of the obtained compound are shown below. ¹H-NMR (400 MHz, CDCl₃) δ 8.18 (s, 1H), 7.77 (s, 1H), 7.64 (d, J=8.2 Hz, 2H), 7.44-7.41 (m, 3H), 7.32 (t, J=9.1 Hz, 2H), 7.09 (s, 1H), 5.76 (s, 2H), 3.84 (s, 2H), 1.97 (s, 3H), ¹³C-NMR (100 MHz, CDCl₃) δ ¹³C-NMR (101 MHz, CDCl₃) δ 170.7, 169.7, 142.4, 135.6, 133.3, 128.6, 127.5, 127.3, 126.4, 124.0, 122.1, 117.2, 111.5, 107.6, 79.4, 31.0, 20.5. TOF-MS m/z 346.1 [M+Na]⁺

[Examination of Effect of 5-pH-IAA AM Ester in Embryos]

An egg-planting embryo expressing AtTIR1 (F79G)::mRuby and AID*::GFP was placed in a droplet of an M9 buffer including 50 μM 5-Ph-IAA-AM, and then the reporter intensity was monitored (see FIG. 4D). It was confirmed that in contrast to the embryos treated with DMSO (see FIG. 4E), 50 μM 5-Ph-IAA-AM effectively caused the rapid removal of the AID*::GFP reporter. In contrast to 5-Ph-IAA, 5-Ph-IAA-AM caused the rapid degradation in all embryos (see FIG. 4F). These results indicate that 5-Ph-IAA-AM is an excellent ligand that targets embryonic proteins.

### INDUSTRIAL APPLICABILITY

According to the auxin-degron system of the present invention, it is possible to efficiently control the degradation of a target protein.

What is claimed is:

1. An auxin-degron system kit controlling degradation of a target protein in a non-plant-derived eukaryotic cell, the kit comprising:
a first nucleic acid encoding a mutant TIR1 family protein that has a mutation at an auxin-binding site;
an auxin analog having an affinity for the mutant TIR1 family protein; and
a second nucleic acid encoding a degradation tag that includes at least a partial Aux/IAA family protein and has an affinity for a complex of the mutant TIR1 family protein and the auxin analog,
wherein the mutant TIR1 family protein is a protein that consists of a sequence including any one amino acid sequence of the following (a) to (c), and binds to the degradation tag through the complex with the auxin analog, leading to degradation of the target protein,
(a) an amino acid sequence in which an amino acid at position 79 of an amino acid sequence set forth in SEQ ID NO: 1 is glycine,
(b) an amino acid sequence in which one to several amino acids are deleted, inserted, substituted, or added in a site other than the amino acid at position 79 of (a), and
(c) an amino acid sequence having 80% or more identity in a site other than the amino acid at position 79 of (a), and
the auxin analog is a compound represented by General Formula (I), 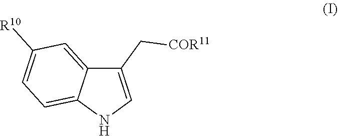
(in General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms).

2. The kit according to claim 1, further comprising:
a third nucleic acid encoding a target protein, which is linked upstream or downstream of the second nucleic acid.

3. The kit according to claim 1,
wherein the mutant TIR1 family protein is an Arabidopsis thaliana-derived protein.

4. The kit according to claim 1,
wherein the mutant TIR1 family protein is a protein in which F at position 79 of AtTIR1 has been mutated into A, G, or S.

5. The kit according to claim 1,
further comprising:
a non-plant-derived eukaryotic cell or non-human animal, having the first nucleic acid on a chromosome.

6. The kit according to claim 5,
wherein the non-plant-derived eukaryotic cell or non-human animal further has a chromosome including a second nucleic acid encoding a degradation tag that includes at least a partial Aux/IAA family protein and has an affinity for a complex of the mutant TIR1 family protein and the auxin analog, and a third nucleic acid encoding a target protein, which is linked upstream or downstream of the second nucleic acid.

7. A method for degrading a target protein, the method using the kit according to any one of claims 1 to 6.

8. An inducer for degradation of a target protein, used in an auxin-degron system controlling degradation of a target protein in a non-plant-derived eukaryotic cell, the inducer comprising:
a compound represented by General Formula (I), 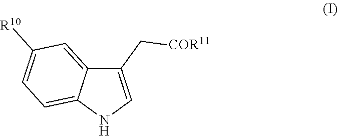
(in General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms).

9. A compound represented by General Formula (I), 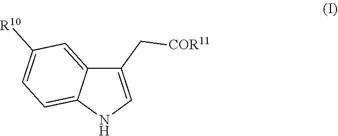
(in General Formula (I), R¹⁰ is a cyclic aliphatic hydrocarbon group in which a substituent may be present and some of carbon atoms constituting a ring may be substituted with heteroatoms, or an aromatic hydrocarbon group in which a substituent may be present and some of the carbon atoms constituting a ring may be substituted with heteroatoms, R¹¹ is —O—(CH₂)ₙ₁—O—C(═O)R¹², —OR¹², or —N(R¹³)₂, n1 is an integer of 1 to 6, R¹² is an alkyl group having 1 to 6 carbon atoms, and a plurality of R¹³'s are each independently a hydrogen atom or an alkyl group having 1 to 6 carbon atoms).
